(19) United States
(12) Patent Application Publication
(10) Pub. No.: US 2026/0183280 A1
(43) Pub. Date: Jul. 2, 2026

(54) LIQUID COMPOSITIONS OF POSACONAZOLE, PREPARATION METHODS AND USES THEREOF

(71) Applicant: Hefei Cosource Pharmaceuticals Co., Ltd, Hefei (CN)

(72) Inventors: Yihua WANG, Hefei, Anhui Province (CN); Hongzhang SUN, Hefei, Anhui Province (CN); Dengjun CHEN, Hefei, Anhui Province (CN); Yuting JIN, Hefei, Anhui Province (CN); Jieyong CAO, Hefei, Anhui Province (CN); Liuyu SHI, Hefei, Anhui Province (CN); Xujun FANG, Hefei, Anhui Province (CN); Jiandong LIAO, Hefei, Anhui Province (CN); Jiahui WANG, Hefei, Anhui Province (CN)

(21) Appl. No.: 19/437,123

(22) Filed: Dec. 30, 2025

(30) Foreign Application Priority Data
Dec. 31, 2024 (CN) 202411995134.1

(51) Int. Cl. A61K 31/496; A61K 47/02; A61K 47/18; A61K 47/20; A61K 47/26; A61K 47/38; A61K 47/40

(52) U.S. Cl. CPC A61K 31/496; A61K 47/02; A61K 47/183; A61K 47/20; A61K 47/26; A61K 47/38; A61K 47/40

(57) ABSTRACT

The present disclosure provides a liquid composition comprising posaconazole, and a preparation method and use thereof. The liquid composition comprises posaconazole, a cyclodextrin, a dilution stabilizer, an antioxidant, and an acidic pH regulator. The dilution stabilizer comprises hydroxypropyl cellulose and/or hydroxypropyl methylcellulose, and the cyclodextrin is present in an amount of 25% to 45% (w/v), based on the total volume of the liquid composition. The liquid composition can increase an oral bioavailability of posaconazole, mitigate an impact of food on bioavailability of posaconazole, and improve patient compliance.

### CROSS-REFERENCE TO RELATED APPLICATIONS

The present application claims the benefit and priority to the Chinese patent application No. 202411995134.1 filed before the China national intellectual property administration on Dec. 31, 2024, the content of which is incorporated herein by reference in its entirety.

### TECHNICAL FIELD

The present disclosure belongs to the field of pharmaceutical formulations, and specifically relates to a liquid composition comprising posaconazole, and a preparation method and use thereof.

### BACKGROUND

The systemic exposure of an oral suspension of posaconazole is significantly affected by food and various gastrointestinal conditions, including pH and motility. In most patients, the plasma concentration of posaconazole is extremely low with an unstable absorption profile. The oral suspension of posaconazole requires multiple daily dosing, such as 200 mg each time and three times daily, and must be administered with a high-fat meal to ensure adequate oral absorption and systemic pharmacokinetic (PK) exposure. However, it is difficult for patients requiring antifungal prophylaxis to ensure a sufficient high-fat dietary intake to enhance the efficacy of the posaconazole suspension due to chemotherapy-induced severe nausea, vomiting, mucositis, and/or diarrhea. These unstable absorption issues prompted the development of a subsequently marketed sustained-release tablet, which has proven to have a higher but still unstable exposure profile. For instance, following administration of 300 mg of the posaconazole tablet, the maximum concentrations achieved after single administration (fasted state) and steady-state administration (fed state) was 614 ng/mL and 2764 ng/mL, respectively (Study P07783). The FDA recommends that the tablet should still be administered with food. In addition, the tablets need to increase a loading dose on Day 1 to achieve a higher concentration of posaconazole more quickly. That is, the tablet is greatly affected by food and requires a controlled loading dose.

### SUMMARY

A first aspect of the present disclosure provides a liquid composition comprising posaconazole, a cyclodextrin, a dilution stabilizer, an antioxidant, an acidic pH regulator and water, wherein the dilution stabilizer comprises hydroxypropyl cellulose and/or hydroxypropyl methylcellulose, and the cyclodextrin is present in an amount of 25% to 45% (w/v), based on the total volume of the liquid composition.

A second aspect of the present disclosure provides a method for preparing the liquid composition as described in the first aspect, comprising: mixing the cyclodextrin with an aqueous solvent to obtain a first mixture; mixing posaconazole, the acidic pH regulator and the antioxidant with the first mixture to obtain a second mixture; mixing the dilution stabilizer with the second mixture to obtain a third mixture; and adding an aqueous solvent to the third mixture to make up the volume.

A third aspect of the present disclosure provides use of the liquid composition as described in the first aspect or the liquid composition as prepared by the method described in the second aspect in the preparation of a medicament for treating, ameliorating, or preventing a fungal infection.

A fourth aspect of the present disclosure provides a method for treating, ameliorating, or preventing a fungal infection, comprising administering to a subject in need thereof the liquid composition as described in the first aspect or the liquid composition as prepared by the method described in the second aspect.

### BRIEF DESCRIPTION OF THE DRAWINGS

FIG. 1 shows the dissolution profiles of posaconazole in the liquid compositions of Examples 1 and 2 of the present disclosure as a function of dissolution time.

FIG. 2 shows the dissolution profiles of posaconazole in the liquid compositions of Examples 3 to 6, and Comparative Examples 2, 4 and 5 of the present disclosure as a function of dissolution time.

FIG. 3 shows the dissolution profiles of posaconazole in the liquid compositions of Examples 7 to 9, 13 and 15 of the present disclosure as a function of dissolution time.

FIG. 4 shows XRD patterns of precipitates obtained from the dissolution determination of the liquid compositions of Examples 3 to 6, and Comparative Examples 2, 4 and 5 of the present disclosure and posaconazole.

FIG. 5 shows XRD patterns of precipitates obtained from the dissolution determination of the liquid compositions of Examples 1, 5, 7 to 14, and Comparative Example 3 of the present disclosure and posaconazole.

### DETAILED DESCRIPTION OF THE INVENTION

The present disclosure is further illustrated below with reference to specific embodiments. It should be understood that these specific embodiments are provided merely for illustrative purposes and are not intended to limit the scope of the present disclosure.

A first aspect of the present disclosure provides a liquid composition comprising posaconazole, a cyclodextrin, a dilution stabilizer, an antioxidant, an acidic pH regulator and water, wherein the dilution stabilizer comprises hydroxypropyl cellulose and/or hydroxypropyl methylcellulose, and the cyclodextrin is present in an amount of 25% to 45% (w/v), based on the total volume of the liquid composition.

In the present disclosure, the combination of the dilution stabilizer with the cyclodextrin can improve the dissolution of posaconazole in different pH environments (e.g., those simulating intestinal pH fluctuations), inhibit the precipitation of posaconazole in crystalline form, and maintain posaconazole in a dissolved or amorphous state within the intestinal tract, thereby ensuring the absorption of posaconazole within the intestinal tract, improving the bioavailability of posaconazole, and mitigating the impact of food on the bioavailability of posaconazole.

The liquid composition of the present disclosure may additionally exhibit one or more of the following beneficial effects: small inter-individual variability, no need for a loading dose, absence of an organic solvent, and improved patient compliance.

In some embodiments of the present disclosure, the liquid composition may be an oral formulation, for example an oral solution.

In some embodiments of the present disclosure, the dilution stabilizer may comprise hydroxypropyl cellulose and/or hydroxypropyl methylcellulose or be selected from the group consisting of hydroxypropyl cellulose and hydroxypropyl methylcellulose. The dilution stabilizer in the liquid composition can adjust the viscosity of the liquid composition and improve the dissolution stability after dilution of the liquid composition, such as the dissolution stability following clinical administration. In particular, the selection of hydroxypropyl cellulose and/or hydroxypropyl methylcellulose in combination with the cyclodextrin can further improve the bioavailability of posaconazole in the liquid composition in different pH environments, and mitigate the impact of food on the bioavailability of posaconazole.

In some embodiments of the present disclosure, the cyclodextrin is present in an amount of 25% to 45% (w/v), for example, 25%, 26%, 27%, 28%, 28.5%, 29%, 29.5%, 30%, 30.5%, 31%, 31.5%, 32%, 32.5%, 33%, 34%, 35%, 36%, 37%, 38%, 39%, 40%, 40.5%, 41%, 42%, 43%, 44%, 45% (w/v) and so on, based on the total volume of the liquid composition. As a result, it is possible to allow the active ingredient posaconazole to have a high dissolution in different pH environments, thereby improving the bioavailability during the release phase.

In some embodiments of the present disclosure, a mass ratio of posaconazole to the dilution stabilizer is 1:(1 to 7.5), for example, 1:1, 1:1.25, 1:1.5, 1:1.75, 1:2, 1:2.25, 1:2.5, 1:2.75, 1:3, 1:3.25, 1:3.5, 1:3.75, 1:4, 1:4.25, 1:4.5, 1:4.75, 1:5, 1:5.25, 1:5.5, 1:5.75, 1:6, 1:6.25, 1:6.5, 1:6.75, 1:7, 1:7.25, 1:7.5 and so on; and/or a mass ratio of posaconazole to the cyclodextrin in the liquid composition is 1:(10 to 22.5), for example, 1:10, 1:10.5, 1:11, 1:11.5, 1:12, 1:12.5, 1:13, 1:13.5, 1:14, 1:14.5, 1:15, 1:15.5, 1:16, 1:16.5, 1:17, 1:17.5, 1:18, 1:18.5, 1:19, 1:19.5, 1:20, 1:20.5, 1:21, 1:21.5, 1:22, 1:22.5 and so on. The amounts of posaconazole and the dilution stabilizer within the specified range further facilitate posaconazole dissolved from the liquid composition to exist in a dissolved state or in amorphous form, instead of precipitating in crystalline form, thereby improving the dissolution stability and bioavailability of posaconazole during the release phase and mitigating the impact of food on bioavailability. The cyclodextrin in the liquid composition can increase the dissolution and solubility of the active ingredient posaconazole in different pH environments, and in particular, the content of the cyclodextrin within the specified range further facilitates achieving a higher dissolution of posaconazole in different pH environments. Still further, when the amounts of posaconazole, dilution stabilizer and cyclodextrin simultaneously fall within the specified ranges, it is more conducive to improving the dissolution of posaconazole in different pH environments, and also making the liquid composition in a supersaturated state, which enables posaconazole dissolved from the liquid composition in different pH environments to exist in a dissolved state or in amorphous form, and maintain the higher dissolution and dissolution stability during the release phase, thereby further increasing the bioavailability of posaconazole and mitigating the impact of food on the bioavailability thereof. In some specific embodiments, the mass ratio of posaconazole to the dilution stabilizer is also 1:(1.5 to 4), or 1:(2 to 3). In some specific embodiments, the mass ratio of posaconazole to the cyclodextrin is also 1:(11 to 20), or 1:(12.5 to 17.5).

In some embodiments of the present disclosure, the cyclodextrin includes β-cyclodextrin and/or a derivative of β-cyclodextrin. In some specific embodiments, the cyclodextrin may include one or more of hydroxypropyl-β-cyclodextrin, sulfobutyl ether-β-cyclodextrin, and methyl-β-cyclodextrin.

In some embodiments of the present disclosure, the liquid composition may further comprise water, and be free of an organic solvent. This enables the liquid composition to provide the better dissolution characteristics and absorption efficacy of posaconazole, and improve patient compliance.

In some embodiments of the present disclosure, the antioxidant may comprise one or more of methionine, sodium metabisulfite, sodium bisulfite, proline, and glycine. In some specific embodiments, the antioxidant may comprise methionine. In some specific embodiments, the antioxidant may comprise sodium metabisulfite and/or sodium bisulfite. In other embodiments, the antioxidant may comprise methionine and one or more selected from sodium metabisulfite, sodium bisulfite, proline, and glycine. For example, the antioxidant may be selected from a combination of methionine and sodium metabisulfite, a combination of methionine and sodium bisulfite, a combination of methionine and proline, a combination of methionine and glycine, and so on. The antioxidant in the liquid composition may consume oxygen in the liquid composition, thereby inhibiting product deterioration caused by oxidation, and enhancing the stability of posaconazole in the liquid composition. In particular, the use of methionine as an antioxidant not only improves the stability of the liquid composition, but also is non-toxic.

In some embodiments of the present disclosure, the acidic pH regulator may be a commonly-used acidic pH regulator in the pharmaceutical art. For example, the acidic pH regulator may include, but is not limited to, one or more of hydrochloric acid, sulfuric acid, phosphoric acid, and maleic acid. In some specific embodiments, the acidic pH regulator may include hydrochloric acid and/or sulfuric acid. The acidic pH regulator in the liquid composition may adjust the solubility of posaconazole in the liquid composition.

In some embodiments of the present disclosure, the pH value of the liquid composition may be 1.5 to 4, e.g., 1.5, 2, 2.5, 3, 3.5, 4, and so on. Therefore, posaconazole may have good dissolution and solubility in the liquid composition. In some specific embodiments, the pH value of the liquid composition may further be 2 to 3.

In some embodiments of the present disclosure, based on the total volume of the liquid composition, posaconazole may be present in an amount of 1% to 2.7% (w/v), the cyclodextrin may be present in an amount of 25% to 45% (w/v), the dilution stabilizer may be present in an amount of 2% to 15% (w/v), the antioxidant may account for 0.05% to 2% (w/v), and the acidic pH regulator may be present in an amount of 1.5% to 4% (w/v). For example, based on the total volume of the liquid composition, posaconazole may be present in an amount of 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7% (w/v), and so on; cyclodextrin may be present in an amount of 25%, 26%, 27%, 28%, 28.5%, 29%, 29.5%, 30%, 30.5%, 31%, 31.5%, 32%, 32.5%, 33%, 33.5%, 34%, 34.5%, 35%, 36%, 37%, 38%, 39%, 40%, 40.5%, 41%, 42%, 43%, 44%, 45% (w/v), and so on; the dilution stabilizer may be present in an amount of 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15% (w/v), and so on; the antioxidant may be present in an amount of 0.05%, 0.1%, 0.2%, 0.3%, 0.5%, 0.8%, 1%, 1.2%, 1.5%, 1.8%, 2% (w/v), and so on; and the acidic pH regulator may be present in an amount of 1.5%, 2%, 2.5%, 3%, 3.5%, 4% (w/v), and so on. In some specific embodiments, based on the total volume of the liquid composition, posaconazole may be present in an amount of 1.5% to 2.5% (w/v), or even 1.5% to 2.0% (w/v). In some specific embodiments, based on the total volume of the liquid composition, the cyclodextrin may be present in an amount of 25% to 40% (w/v), or even 25% to 35% (w/v). In some specific embodiments, based on the total volume of the liquid composition, the dilution stabilizer may be present in an amount of 3% to 8% (w/v), or even 4% to 6% (w/v). In some specific embodiments, based on the total volume of the liquid composition, the antioxidant may be present in an amount of 0.05% to 1% (w/v), or even 0.05% to 0.3% (w/v). Therefore, the liquid composition of the present disclosure exhibits at least one of the following beneficial effects: improved bioavailability of posaconazole, reduced susceptibility to the impact of food on bioavailability; small inter-individual variability; no need for a loading dose; and absence of an organic solvent.

In some embodiments of the present disclosure, the liquid composition may further comprise one or more of a flavoring agent, a preservative, and a chelating agent. The flavoring agent may improve the palatability of the liquid composition and further increase patient compliance. The preservative is beneficial for inhibiting bacterial growth in the liquid composition. The chelating agent can chelate metal ions in the liquid composition, and further enhance the stability of posaconazole in the liquid composition.

In some embodiments of the present disclosure, the flavoring agent may comprise a sweetener and/or a flavor. The flavoring agent may be a commonly-used flavoring agent in the pharmaceutical art. For example, the flavoring agent includes, but is not limited to, a sweetener and/or a flavor. In some specific embodiments, the sweetener includes, but is not limited to, one or more of sucralose, acesulfame potassium, stevioside, sodium cyclamate, aspartame, saccharin sodium, and xylitol. In some specific embodiments, the flavor includes, but is not limited to, one or more of sweet orange flavor, banana flavor, orange flavor, strawberry flavor, and mango flavor.

In some embodiments of the present disclosure, the preservative may comprise one or more of methylparaben, ethylparaben, and propylparaben. The preservative may be selected from one or more of commonly-used preservatives in the pharmaceutical art. For example, the commonly-used preservative includes, but is not limited to, methylparaben, ethylparaben, and propylparaben.

In some embodiments of the present disclosure, the chelating agent may comprise one or more of citric acid, malic acid, benzoic acid, edetic acid, and sodium edetate. The chelating agent may be selected from one or more of commonly-used chelating agents in the pharmaceutical art. For example, the commonly-used chelating agent includes, but is not limited to, citric acid, malic acid, benzoic acid, edetic acid, and sodium edetate.

In some embodiments of the present disclosure, based on the total volume of the liquid composition, the flavoring agent may be present in an amount of 0.1% to 10% (w/v), and/or the preservative may be present in an amount of 0.01% to 1% (w/v), and/or the chelating agent may be present in an amount of 0.01% to 0.1% (w/v). For example, when the liquid composition further comprises the flavoring agent, the flavoring agent may be present in an amount of 0%, 0.1%, 0.2%, 0.4%, 0.5%, 0.6%, 1%, 2%, 3%, 4%, 5%, 6%, 8%, 9% 10% (w/v), and so on. For another example, when the liquid composition further comprises the preservative, the preservative may be present in an amount of 0.01%, 0.02%, 0.05%, 0.08%, 0.1%, 0.2%, 0.5%, 0.8%, 1% (w/v), and so on. For another example, when the liquid composition further comprises the chelating agent, the chelating agent may be present in an amount of 0.01%, 0.015%, 0.02%, 0.025%, 0.03%, 0.05%, 0.08%, 0.1% (w/v), and so on.

In some embodiments of the present disclosure, the liquid composition may be an oral solution. The liquid composition may be used for treating, ameliorating, or preventing a fungal infection.

A second aspect of the present disclosure provides a method for preparing the liquid composition as described in the first aspect, comprising: mixing the cyclodextrin with an aqueous solvent to obtain a first mixture; mixing posaconazole, the acidic pH regulator and the antioxidant with the first mixture to obtain a second mixture; mixing the dilution stabilizer with the second mixture to obtain a third mixture; and adding the aqueous solvent to the third mixture to make up the volume.

In some embodiments, the mixing operations in each step may be performed independently under stirring conditions. If necessary, the mixing operations in each step may also be performed under heating conditions to facilitate dissolution of each component. For example, posaconazole, the acidic pH regulator and the antioxidant may be mixed with the first mixture under heating and stirring conditions.

In some embodiments, the aqueous solvent may be water, and further free of an organic solvent.

In some embodiments of the present disclosure, the liquid composition may further comprise one or more of the flavoring agent, the preservative, and the chelating agent. Depending on practical requirements, a desired amount of the flavoring agent, preservative, chelating agent, or any combination thereof may be optionally added during at least one of the mixing steps. For example, in some specific embodiments, the chelating agent and/or the preservative may be added while mixing cyclodextrin with the aqueous solvent. In some specific embodiments, the flavoring agent may be mixed with the third mixture prior to the step of adding the aqueous solvent to make up the volume.

In some embodiments of the present disclosure, the preparation and storage of the liquid composition may be carried out under a protective atmosphere. In some specific embodiments, the protective atmosphere is a non-oxidizing atmosphere, such as nitrogen atmosphere.

A third aspect of the present disclosure provides use of the liquid composition described in the first aspect or the liquid composition prepared by the method described in the second aspect in the preparation of a medicament for treating, ameliorating, or preventing a fungal infection.

A fourth aspect of the present disclosure provides a method for treating, ameliorating, or preventing a fungal infection, comprising administering to a subject in need thereof the liquid composition described in the first aspect or the liquid composition prepared by the method described in the second aspect.

In some embodiments, the fungal infection is selected from one or more of the following diseases, disorders or conditions: oropharyngeal or esophageal candidiasis; refractory oropharyngeal or esophageal candidiasis; invasive aspergillosis, candidiasis, fusaridiosis, scedosporiosis, infections caused by dimorphic fungi, zygomycosis, and invasive infections caused by rare fungi and yeasts; invasive fungal diseases in patients who are not responsive or intolerant to other therapies; candidiasis and invasive mycotic infections in patients undergoing intensive chemotherapies and/or radiotherapies due to hematological malignancies, bone marrow or peripheral stem cell transplantation pretreatment regimens, and in patients receiving combined immunosuppressive therapies for the treatment of acute or chronic graft-versus-host disease or for the prevention of solid organ transplant rejection; Chagas's disease; and leishmaniasis.

The liquid composition of the present disclosure exhibits one or more of the following beneficial effects: improved dissolution of posaconazole in different pH environments (e.g., those simulating intestinal pH fluctuations), inhibiting the precipitation of posaconazole in crystalline form, enhanced bioavailability of posaconazole, reduced impact of food on the bioavailability, small inter-individual variability; no need for a loading dose; absence of an organic solvent; improved patient compliance; and providing additional options for patients unable to take oral enteric-coated tablets.

### EXAMPLES

The present disclosure will be illustrated in further detail below with reference to the following specific Examples. Various modifications can be made to these examples to obtain other embodiments without departing from the scope or spirit of the present disclosure. Therefore, the following examples are illustrative only and not intended to be limiting.

Unless otherwise specified, all numbers used in the Specification and Claims to represent feature sizes, quantities, and physicochemical properties should be understood as being modified by the term “about” in all cases. Therefore, unless otherwise stated to the contrary, the numerical parameters listed in the foregoing Specification and the appended claims are approximations, and those skilled in the art can utilize the teachings disclosed herein to seek the desired characteristics, and appropriately change these approximations. The use of a numerical range represented by endpoints includes all numbers within said range and any range within said range, for example, 1 to 5 includes 1, 1.1, 1.3, 1.5, 2, 2.75, 3, 3.80, 4, and 5, etc.

The drugs or reagents used in the present disclosure are all conventional commercially-available products, unless otherwise specified.

### General Determination Methods:

### (1) Dissolution Determination Method:

Unless otherwise specified, the dissolution of an oral solution of posaconazole is determined using the following method.

Dissolution: A 5 mL of the sample was taken and placed in 500 mL of 0.2 mol/L phosphate buffer at pH 6.8 as the dissolution medium. The dissolution determination was performed at a rotation speed of 50 rpm and a medium temperature of 37° C. in accordance with the Dissolution and Release Determination Method (Method II, General Rules 0931, Part IV of the Chinese Pharmacopoeia 2020 Edition). Appropriate volumes of the solution were taken at specified time intervals (i.e., 10 min, 30 min, 60 min, 120 min, 240 min, 360 min and 480 min), and centrifuged at 12,000 rpm for 5 min, and the resulting supernatant was taken and diluted fivefold with methanol to prepare a test solution. An appropriate amount of posaconazole reference standard was taken, dissolved and diluted in methanol-hydrochloric acid (100:0.4) to prepare a stock solution containing approximately 0.4 mg of posaconazole per ml. Subsequently, 2 mL of the stock solution was accurately weighted and placed in a 20 mL volumetric flask, and diluted to the mark with the dissolution medium to obtain a reference standard solution. The content of posaconazole was determined by high-performance liquid chromatography in accordance with General Rules 0512, Part IV of the Chinese Pharmacopoeia 2020 Edition. Packing material: octadecylsilane-bonded silica gel; mobile phase: aqueous phosphoric acid solution [prepared by adding water to 1.5 ml of phosphoric acid to 640 ml and stirring to mix uniformly]-acetonitrile (640:36); flow rate: 1.0 ml/min; column temperature: 30° C.; and detection wavelength: 254 nm. The number of theoretical plates calculated based on posaconazole should not be less than 2000. The dissolution amount of posaconazole in each vessel at different times was calculated using the external standard method.

### (2) Content Determination Method:

Content: The content was determined by high-performance liquid chromatography in accordance with General Rules 0512, Part IV of the Chinese Pharmacopoeia 2020 Edition.

Test solution: an appropriate amount (about 1 g) of the sample was taken, accurately weighed and placed in a 100 ml volumetric flask, and an appropriate amount of methanol-water-hydrochloric acid (100:100:0.2) was added, shaken to dissolve, and diluted to the mark with a solvent, shaken to mix uniformly, and filtered, and the subsequent filtrate was taken.

Reference Standard Solution: an appropriate amount of posaconazole reference standard was taken, accurately weighed, then dissolved and diluted in methanol to prepare a stock solution containing approximately 1 mg of posaconazole per ml. Subsequently, 5 mL of the stock solution was accurately weighted and placed in a 25 mL volumetric flask, and diluted to the mark with methanol-water-hydrochloric acid (100:100:0.2) to obtain a reference standard solution.

Chromatographic Conditions: Packing material: octadecylsilane-bonded silica gel; mobile phase: aqueous phosphoric acid solution [prepared by adding water to 1.5 ml of phosphoric acid to 640 ml and stirring to mix uniformly]-acetonitrile (640:36); flow rate: 1.0 ml/min; detection wavelength: 254 nm; column temperature: 30° C.; and injection volume: 10 μL.

System Suitability Requirement: The number of theoretical plates (N) calculated based on the peak of posaconazole shall not be less than 3000.

Measurement Method: The test solution and the reference standard solution were precisely measured, and injected into the liquid chromatograph, respectively. Chromatograms were recorded. The content was calculated using the external standard method based on peak area.

### (3) Related Substance Determination Method:

Unless otherwise specified, an impurity of posaconazole was determined using the following high-performance liquid chromatographic conditions:

Related Substance: A related substance was determined by high-performance liquid chromatography in accordance with General Rules 0512, Part IV of the Chinese Pharmacopoeia 2020 Edition. Packing material: octadecylsilane-bonded silica gel; mobile phase: acetonitrile—0.05 mol/L ammonium acetate solution (pH adjusted to 4.0 with acetic acid); a gradient elution; flow rate: 1.0 ml/min.

The gradient elution procedure was shown in Table 1:

TABLE 1

| Time (min) | % B |
|---|---|
| 0.01 | 5.0 |
| 3.00 | 35.0 |
| 4.00 | 35.0 |
| 17.00 | 60.0 |
| 18.00 | 60.0 |
| 35.00 | 90.0 |
| 36.00 | 90.0 |
| 40.00 | 5.0 |
| 50.00 | 5.0 |

The detection wavelength was 262 nm. The number of theoretical plates calculated based on the peak of posaconazole was not less than 3000, and the resolution between adjacent impurity peaks shall meet the requirements. An appropriate amount of the oral solution of posaconazole was taken, and diluted with 50% acetonitrile to prepare a solution containing 1.5 mg/mL of posaconazole. The solution was centrifuged, and the supernatant was injected into the liquid chromatograph. The chromatogram was recorded. The content was calculated as the percentage of peak area using the area normalization method.

### (4) Crystal Form Determination Method:

Unless otherwise specified, the crystal form of posaconazole precipitates was determined using the following method.

The precipitates were collected following dissolution completion, combined and centrifugated at 12000 rpm for 5 min. The sediments were taken, air-dried and ground to fine powders, and the crystal form was determined using a DX-27mini X powder diffractometer.

### Sources of Drugs and Reagents:

Unless otherwise specified, the drugs and reagents used in the following Examples and Comparative Examples are all commercially available products, and the term “Percentage (w/v %)” indicates the percentage of the mass (g) of each component based on the total volume (ml) of the liquid composition, i.e., weight to volume percentage.
Posaconazole: purchased from Wuhan Ruisheng Pharmaceuticals Co., Ltd., Lot No. C046-2307001;
Hydroxypropyl-β-cyclodextrin: purchased from Shandong Binzhou Zhiyuan Biotechnology Co., Ltd., Lot No. HP20240315;
Sulfobutyl ether-β-cyclodextrin, supplied by Ron Pharm (Shanghai) Co., Ltd., Manufacturer: Cyclolab Cyclodextrin Research & Development Laboratory Ltd., Lot No. 47K150521;
Hydroxypropyl methylcellulose, purchased from Anhui Sunhere Pharmaceutical Excipients Co., Ltd., Lot No. 240203;
Hydroxypropyl cellulose, purchased from Shanghai Sunjoy Pharmaceutical Excipients Co., Ltd., Lot No. 64328.

### Comparative Example 1

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.03 | g | 0.02% |
| Hydroxypropyl methylcellulose | 7.5 | g | 5.00% |
| Acesulfame potassium | 0.15 | g | 0.10% |
| Orange flavor | 0.45 | g | 0.30% |
| 10% sulfuric acid | 1.5 | g | |
| aqueous solution | | | |

| | |
|---|---|
| Water | To 150 ml |

Preparation Method: Hydroxypropyl-β-cyclodextrin and disodium edetate were weighed and dissolved in water with stirring, and then posaconazole and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and orange flavor were added, and stirred to dissolve. The resulting solution was adjusted to pH of 2.5 with 1% sulfuric acid or 1 mol/L NaOH solution, and water was added to make up the volume.

### Comparative Example 2

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.15 | g | 0.10% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | 0.20% |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (commercially available hydrochloric acid, with a concentration of 36% to 38%) were added. The mixture was placed in a 60° C. water bath and stirred to dissolve the above components. The resulting mixture was cooled, and then sucralose and cherry flavor were added and stirred to dissolve. The resulting solution was adjusted to pH of 2.5 with diluted hydrochloric acid or 1 mol/L NaOH solution, and water was added to make up the volume.

### Comparative Example 3

| Component | Formula amount | | Percentage (w/v %) |
|---|---|---|---|
| Posaconazole | 3 | g | 2.00% |
| Hydroxypropyl-β-cyclodextrin | 75 | g | 50.00% |
| Disodium edetate | 0.15 | g | 0.10% |
| Ascorbic acid | 0.45 | g | 0.3 |
| Hydroxypropyl cellulose | 3 | g | 2.00% |
| Sodium metabisulfite | 0.3 | g | 0.20% |
| Hydrochloric acid | 0.3 | g | 0.20% |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite, ascorbic acid and hydrochloric acid (the same as that in Comparative Example 2) were added. The mixture was placed in a 60° C. water bath and stirred to dissolve the above components. Hydroxypropyl cellulose was added and stirred to dissolve. The resulting solution was adjusted to pH of 4.0 with diluted hydrochloric acid or 1 mol/L NaOH solution, and water was added to make up the volume.

### Comparative Example 4

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.15 | g | 0.10% |
| Povidone K30 | 22.5 | g | 15.00% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. The resulting mixture was cooled, and then povidone K30 was added and stirred to dissolve. Sucralose and cherry flavor were added and stirred to dissolve. The resulting solution was adjusted to pH of 2.5 with diluted hydrochloric acid or 1 mol/L NaOH solution, and water was added to make up the volume.

### Comparative Example 5

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.15 | g | 0.10% |
| Polyethylene glycol 4000 | 22.5 | g | 15.00% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. The resulting mixture was cooled, and then polyethylene glycol 4000 was added and stirred to dissolve. Sucralose and cherry flavor were added and stirred to dissolve. The resulting solution was adjusted to pH of 2.5 with diluted hydrochloric acid or 1 mol/L NaOH solution, and water was added to make up the volume.

### Example 1

| Component | Formula amount | | Percentage (w/v %) |
|---|---|---|---|
| Posaconazole | 3 | g | 2.00% |
| Hydroxypropyl-β-cyclodextrin | 37.5 | g | 25.00% |
| Disodium edetate | 0.15 | g | 0.10% |
| Hydroxypropyl cellulose | 3 | g | 2.00% |
| Sodium metabisulfite | 0.3 | g | 0.20% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. Hydroxypropyl cellulose was added and stirred to dissolve. The resulting solution was adjusted to pH of 1.5 with diluted hydrochloric acid or 1 mol/L NaOH solution, and water was added to make up the volume.

### Example 2

| Component | Formula amount | | Percentage (w/v %) |
|---|---|---|---|
| Posaconazole | 3 | g | 2.00% |
| Hydroxypropyl-β-cyclodextrin | 67.5 | g | 45.00% |
| Disodium edetate | 0.15 | g | 0.10% |
| Hydroxypropyl cellulose | 3 | g | 2.00% |
| Sodium metabisulfite | 0.3 | g | 0.20% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. Hydroxypropyl cellulose was added and stirred to dissolve. The resulting solution was adjusted to pH of 4 with diluted hydrochloric acid or 1 mol/L NaOH solution, and water was added to make up the volume.

### Example 3

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.15 | g | 0.10% |
| Hydroxypropyl methylcellulose | 22.5 | g | 15.00% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. Hydroxypropyl methylcellulose was added and stirred to dissolve. The resulting mixture was cooled, and then sucralose and cherry flavor were added and stirred to dissolve. The resulting solution was adjusted to pH of 2.5 with diluted hydrochloric acid or 1 mol/L NaOH solution, and water was added to make up the volume.

### Example 4

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.15 | g | 0.10% |
| Hydroxypropyl methylcellulose | 15 | g | 10.00% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. Hydroxypropyl methylcellulose was added and stirred to dissolve. The resulting mixture was cooled, and then sucralose and cherry flavor were added and stirred to dissolve. The resulting solution was adjusted to pH of 2.5 with diluted hydrochloric acid or 1 mol/L NaOH solution, and water was added to make up the volume.

### Example 5

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.15 | g | 0.10% |
| Hydroxypropyl methylcellulose | 7.5 | g | 5.00% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. Hydroxypropyl methylcellulose was added and stirred to dissolve. The resulting mixture was cooled, and then sucralose and cherry flavor were added and stirred to dissolve. The resulting solution was adjusted to pH of 2.5 with diluted hydrochloric acid or 1 mol/L NaOH solution, and water was added to make up the volume.

### Example 6

| Component | Formula amount | | Percentage (w/v %) |
|---|---|---|---|
| Posaconazole | 3 | g | 2.00% |
| Hydroxypropyl-β-cyclodextrin | 45 | g | 30.00% |
| Disodium edetate | 0.15 | g | 0.10% |
| Hydroxypropyl methylcellulose | 3 | g | 2.00% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. Hydroxypropyl methylcellulose was added and stirred to dissolve. The resulting mixture was cooled, and then sucralose and cherry flavor were added and stirred to dissolve. The resulting solution was adjusted to pH of 2.3 with diluted hydrochloric acid or 1 mol/L NaOH solution, and water was added to make up the volume.

### Example 7

| Component | Formula amount | | Percentage (w/v %) |
|---|---|---|---|
| Posaconazole | 3 | g | 2.00% |
| Hydroxypropyl-β-cyclodextrin | 50 | g | 33.33% |
| Malic acid | 0.06 | g | 0.04% |
| Hydroxypropyl methylcellulose | 7.5 | g | 5.00% |
| Methionine | 0.3 | g | 0.20% |
| Acesulfame potassium | 0.225 | g | 0.15% |
| Sweet orange flavor | 0.45 | g | 0.30% |
| 10% sulfuric acid | 1.4 | g | |
| aqueous solution | | | |

| | |
|---|---|
| Water | To 150 ml |

Preparation Method: Hydroxypropyl-β-cyclodextrin and malic acid were weighed and dissolved in water with stirring, and then posaconazole, methionine and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and sweet orange flavor were added and stirred to dissolve. The resulting solution was adjusted to pH of 3.0 with 1% sulfuric acid or 1 mol/L NaOH solution, and water was added to make up the volume.

### Example 8

| Component | Formula amount | | Percentage (w/v %) |
|---|---|---|---|
| Posaconazole | 3 | g | 2.00% |
| Hydroxypropyl-β-cyclodextrin | 52.5 | g | 35.00% |
| Citric acid | 0.045 | g | 0.03% |
| Hydroxypropyl methylcellulose | 15 | g | 10.00% |
| Methionine | 0.3 | g | 0.20% |
| Propylparaben | 0.15 | g | 0.10% |
| Aspartame | 0.225 | g | 0.15% |
| Honey peach flavor | 0.45 | g | 0.30% |
| Phosphoric acid | 0.3 | g | 0.20% |

| | |
|---|---|
| Water | To 150 ml |

Preparation Method: Hydroxypropyl-β-cyclodextrin and citric acid were weighed and dissolved in water with stirring, and then posaconazole, methionine and phosphoric acid (commercially available phosphoric acid, with a concentration of ≥85%) were added. The mixture was placed in a 60° C. water bath and stirred to dissolve the above components. Hydroxypropyl methylcellulose was added and stirred to dissolve. The resulting mixture was cooled, and then propylparaben, aspartame and honey peach flavor were added and stirred to dissolve. The resulting solution was adjusted to pH of 3.5 with 1% phosphoric acid solution, and water was added to make up the volume.

### Example 9

| | | | Percentage |
|---|---|---|---|
| Component | Formula amount | | (w/v %) |
| Posaconazole | 3 | g | 2.00% |
| Sulfobutyl ether-β-cyclodextrin | 40 | g | 26.67% |
| Hydroxypropyl cellulose | 10 | g | 6.67% |
| Sodium metabisulfite | 0.15 | g | 0.10% |
| Benzoic acid | 0.3 | g | 0.20% |
| Sodium cyclamate | 0.2 | g | 0.13% |
| Banana flavor | 0.5 | g | 0.33% |
| 10% sulfuric acid aqueous solution | 1.4 | g | 0.20% |

| | |
|---|---|
| Water | To 150 ml |

Preparation Method: Sulfobutyl ether-β-cyclodextrin and benzoic acid were weighed and dissolved in water with stirring, and then posaconazole, sodium metabisulfite and 10% sulfuric acid aqueous solution were added. The mixture was placed in a 60° C. water bath and stirred to dissolve the above components. Hydroxypropyl cellulose was added and stirred to dissolve. The resulting mixture was cooled, and then sodium cyclamate and banana flavor were added and stirred to dissolve. The resulting solution was adjusted to pH of 3.0 with 1% sulfuric acid or 1 mol/L NaOH solution, and water was added to make up the volume.

### Example 10

| | | | Percentage |
|---|---|---|---|
| Component | Formula amount | | (w/v %) |
| Posaconazole | 3 | g | 2.00% |
| Hydroxypropyl-β-cyclodextrin | 45 | g | 30.00% |
| Disodium edetate | 0.03 | g | 0.02% |
| Hydroxypropyl methylcellulose | 7.5 | g | 5.00% |
| Methionine | 0.075 | g | 0.05% |
| Acesulfame potassium | 0.15 | g | 0.10% |
| Orange flavor | 0.45 | g | 0.30% |
| 10% sulfuric acid aqueous solution | 1.4 | g | 0.20% |

| | |
|---|---|
| Water | To 150 ml |

Preparation Method: Hydroxypropyl-β-cyclodextrin and disodium edetate were weighed and dissolved in water with stirring, and then posaconazole, methionine and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and orange flavor were added and stirred to dissolve. The resulting solution was adjusted to pH of 2.5 with 1% sulfuric acid or 1 mol/L NaOH solution, and water was added to make up the volume.

### Example 11

| | | | Percentage |
|---|---|---|---|
| Component | Formula amount | | (w/v %) |
| Posaconazole | 3 | g | 2.00% |
| Hydroxypropyl-β-cyclodextrin | 45 | g | 30.00% |
| Disodium edetate | 0.03 | g | 0.02% |
| Hydroxypropyl methylcellulose | 7.5 | g | 5.00% |
| Methionine | 0.3 | g | 0.20% |
| Acesulfame potassium | 0.15 | g | 0.10% |
| Orange flavor | 0.45 | g | 0.30% |
| 10% sulfuric acid aqueous solution | 1.4 | g | 0.20% |

| | |
|---|---|
| Water | To 150 ml |

Preparation Method: Hydroxypropyl-β-cyclodextrin and disodium edetate were weighed and dissolved in water with stirring, and then posaconazole, methionine and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and orange flavor were added and stirred to dissolve. The resulting solution was adjusted to pH of 2.5 with 1% sulfuric acid or 1 mol/L NaOH solution, and water was added to make up the volume.

### Example 12

| | | | Percentage |
|---|---|---|---|
| Component | Formula amount | | (w/v %) |
| Posaconazole | 3 | g | 2.00% |
| Hydroxypropyl-β-cyclodextrin | 45 | g | 30.00% |
| Disodium edetate | 0.03 | g | 0.02% |
| Hydroxypropyl methylcellulose | 7.5 | g | 5.00% |
| Methionine | 0.75 | g | 0.50% |
| Acesulfame potassium | 0.15 | g | 0.10% |
| Orange flavor | 0.45 | g | 0.30% |
| 10% sulfuric acid aqueous solution | 1.4 | g | 0.20% |

| | |
|---|---|
| Water | To 150 ml |

Preparation Method: Hydroxypropyl-β-cyclodextrin and disodium edetate were weighed and dissolved in water with stirring, and then posaconazole, methionine and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and orange flavor were added and stirred to dissolve. The resulting solution was adjusted to pH of 2.5 with 1% sulfuric acid or 1 mol/L NaOH solution, and water was added to make up the volume.

### Example 13

| | | | Percentage |
|---|---|---|---|
| Component | Formula amount | | (w/v %) |
| Posaconazole | 1.5 | g | 1.00% |
| Hydroxypropyl-β-cyclodextrin | 45 | g | 30.00% |
| Disodium edetate | 0.03 | g | 0.02% |
| Hydroxypropyl methylcellulose | 7.5 | g | 5.00% |
| Sodium bisulfite | 0.5 | g | 0.33% |
| Acesulfame potassium | 0.15 | g | 0.10% |
| Orange flavor | 0.45 | g | 0.30% |
| 10% sulfuric acid aqueous solution | 1.4 | g | 0.20% |

| | |
|---|---|
| Water | To 150 ml |

Preparation Method: Hydroxypropyl-β-cyclodextrin and disodium edetate were weighed and dissolved in water with stirring, and then posaconazole, sodium bisulfite and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and orange flavor were added and stirred to dissolve. The resulting solution was adjusted to pH of 2.5 with 1% sulfuric acid or 1 mol/L NaOH solution, and water was added to make up the volume.

### Example 14

| | | | Percentage |
|---|---|---|---|
| Component | Formula amount | | (w/v %) |
| Posaconazole | 4 | g | 2.67% |
| Hydroxypropyl-β-cyclodextrin | 50 | g | 33.33% |
| Disodium edetate | 0.03 | g | 0.02% |
| Hydroxypropyl methylcellulose | 7.5 | g | 5.00% |
| Sodium bisulfite | 0.5 | g | 0.33% |
| Sodium cyclamate | 0.15 | g | 0.10% |
| Cherry flavor | 0.45 | g | 0.30% |
| 10% sulfuric acid aqueous solution | 1.4 | g | 0.20% |

| | |
|---|---|
| Water | To 150 ml |

Preparation Method: Hydroxypropyl-β-cyclodextrin and disodium edetate were weighed and dissolved in water with stirring, and then posaconazole, sodium bisulfite and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then sodium cyclamate and cherry flavor were added and stirred to dissolve. The resulting solution was adjusted to pH of 2.0 with 1% sulfuric acid or 1 mol/L NaOH solution, and water was added to make up the volume.

### Example 15

| | | | Percentage |
|---|---|---|---|
| Component | Formula amount | | (w/v %) |
| Posaconazole | 3 | g | 2.00% |
| Hydroxypropyl-β-cyclodextrin | 45 | g | 30.00% |
| Disodium edetate | 0.15 | g | 0.10% |
| Hydroxypropyl cellulose | 15 | g | 10.00% |
| Sodium metabisulfite | 0.3 | g | 0.20% |
| Sucralose | 0.225 | g | 0.15% |
| Cherry flavor | 0.45 | g | 0.30% |
| Hydrochloric acid | 0.3 | g | |

| | |
|---|---|
| Water | To 150 ml |

Preparation Method: Hydroxypropyl-β-cyclodextrin and disodium edetate were weighed and dissolved in water with stirring, and then posaconazole, sodium metabisulfite and hydrochloric acid (the same as that in Comparative Example 2) were added. The mixture was placed in a 60° C. water bath and stirred to dissolve the above components. Hydroxypropyl cellulose was added and stirred to dissolve. The resulting mixture was cooled, and then sucralose and cherry flavor were added and stirred to dissolve. The resulting solution was adjusted to pH of 2.5 with diluted hydrochloric acid or 1 mol/L NaOH solution, and water was added to make up the volume.

### Example 16

| | | | Percentage |
|---|---|---|---|
| Component | Formula amount | | (w/v %) |
| Posaconazole | 3 | g | 2.00% |
| Hydroxypropyl-β-cyclodextrin | 45 | g | 30.00% |
| Disodium edetate | 0.03 | g | 0.02% |
| Ascorbic acid | 0.45 | g | 0.30% |
| Hydroxypropyl methylcellulose | 7.5 | g | 5.00% |
| Acesulfame potassium | 0.15 | g | 0.10% |
| Orange flavor | 0.45 | g | 0.30% |
| 10% sulfuric acid aqueous solution | 1.5 | g | |

| | |
|---|---|
| Water | To 150 ml |

Preparation Method: Hydroxypropyl-β-cyclodextrin and disodium edetate were weighed and dissolved in water with stirring, and then posaconazole, ascorbic acid and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and orange flavor were added and stirred to dissolve. The resulting solution was adjusted to pH of 2.5 with 1% sulfuric acid or 1 mol/L NaOH solution, and water was added to make up the volume.

### Example 17

| | | | Percentage |
|---|---|---|---|
| Component | Formula amount | | (w/v %) |
| Posaconazole | 3 | g | 2.00% |
| Hydroxypropyl-β-cyclodextrin | 45 | g | 30.00% |
| Disodium edetate | 0.03 | g | 0.02% |
| Thioglycerol | 0.45 | g | 0.30% |
| Hydroxypropyl methylcellulose | 7.5 | g | 5.00% |
| Acesulfame potassium | 0.15 | g | 0.10% |
| Orange flavor | 0.45 | g | 0.30% |
| 10% sulfuric acid aqueous solution | 1.5 | g | |

| | |
|---|---|
| Water | To 150 ml |

Preparation Method: Hydroxypropyl-β-cyclodextrin and disodium edetate were weighed and dissolved in water with stirring, and then posaconazole, thioglycerol and 10% sulfuric acid aqueous solution were added. The mixture was placed in a 60° C. water bath and stirred to dissolve the above components. Hydroxypropyl methylcellulose was added and stirred to dissolve. The resulting mixture was cooled, and then acesulfame potassium and orange flavor were added and stirred to dissolve. The resulting solution was adjusted to pH of 2.5 with 1% sulfuric acid or 1 mol/L NaOH solution, and water was added to make up the volume.

### Test Examples

### 1. Solubility Test of Posaconazole

Refer to the “Content Determination Method” in the aforementioned general determination methods.

### 1) Solubility Test of Posaconazole in Aqueous Solutions Having Different Concentrations of Hydroxypropyl-β-Cyclodextrin and Different pH Values

Hydroxypropyl-β-cyclodextrin was dissolved in water to prepare aqueous solutions with concentrations of 20%, 25%, 30%, 35%, and 40%, respectively. Excess amounts of posaconazole were added to form suspensions. The pH values of the suspensions were adjusted to 2.0, 3.0, and 4.0, respectively, using 1% sulfuric acid solution. The resulting mixtures were heated in a 60° C. water bath, and stirred to dissolve, then cooled to room temperature. The mixtures were left to stand for 48 hours, and then centrifuged at 12,000 rpm for 5 minutes. The supernatants were collected and diluted 100-fold with methanol-water-hydrochloric acid (100:100:0.2). The concentration of posaconazole in the supernatants was determined using the aforementioned content determination method. The saturated solubility of posaconazole was calculated at different hydroxypropyl-β-cyclodextrin concentrations and different pH values, and the results were shown in Table 2.

TABLE 2

| Hydroxypropyl-β- | | Saturated |
|---|---|---|
| cyclodextrin concentration | pH | Solubility (mg/ml) |
| 20% | 2.05 | 16.03 |
| | 3.02 | 9.46 |
| | 4.05 | 7.83 |
| 25% | 2.02 | 19.62 |
| | 2.97 | 11.94 |
| | 3.96 | 10.73 |
| 30% | 2.01 | 27.35 |
| | 3.01 | 20.79 |
| | 3.97 | 14.21 |
| 35% | 2.01 | 26.20 |
| | 3.07 | 21.39 |
| | 4.05 | 19.11 |
| 40% | 2.05 | 36.75 |
| | 3.05 | 23.69 |
| | 4.05 | 21.82 |

Conclusion: Based on the above saturated solubility of posaconazole in aqueous solutions having different concentrations of hydroxypropyl-β-cyclodextrin and different pH values, it was evident that the higher the concentrations of hydroxypropyl-β-cyclodextrin, the greater the saturated solubility of posaconazole, and conversely, the higher the pH value, the lower the saturated solubility of posaconazole. This indicates that hydroxypropyl-β-cyclodextrin can significantly increase the solubility of posaconazole, and that posaconazole can form salts under a strongly acidic condition to increase its solubility.

### 2) Effect of Hydroxypropyl-β-Cyclodextrin Concentration in Liquid Composition on Dissolution of Posaconazole

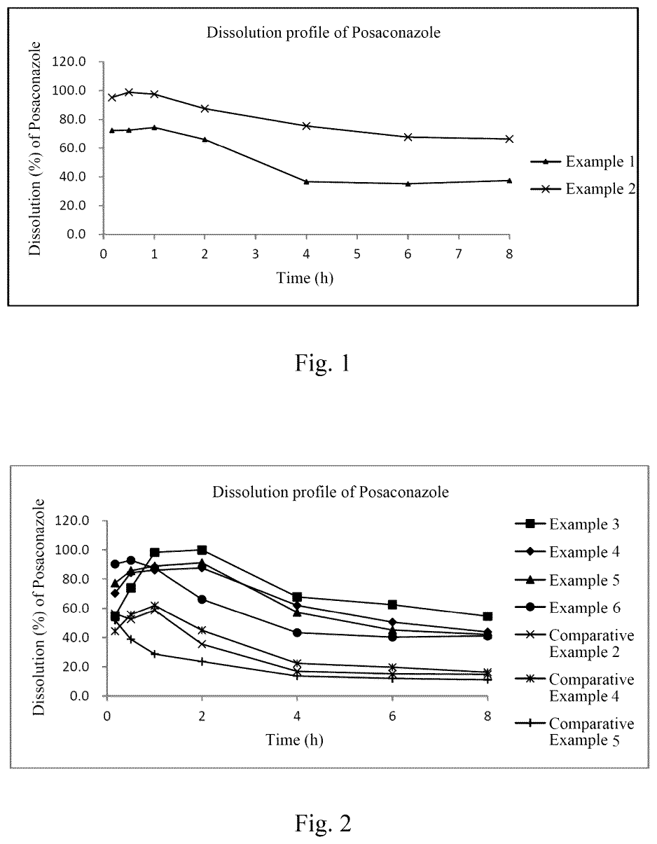

The dissolution of posaconazole at different dissolution times was determined for the liquid compositions of Example 1 and Example 2, respectively, and the results were shown in FIG. 1.

Conclusion: As shown in FIG. 1, a higher amount of hydroxypropyl-β-cyclodextrin in the liquid composition resulted in a greater concentration of posaconazole released in the near-neutral dissolution medium following dilution.

### 3) Effect of Dilution Stabilizer in Liquid Composition on Dissolution of Posaconazole

The dissolution of posaconazole at different dissolution times was determined for the liquid composition of each Example and each Comparative Example.

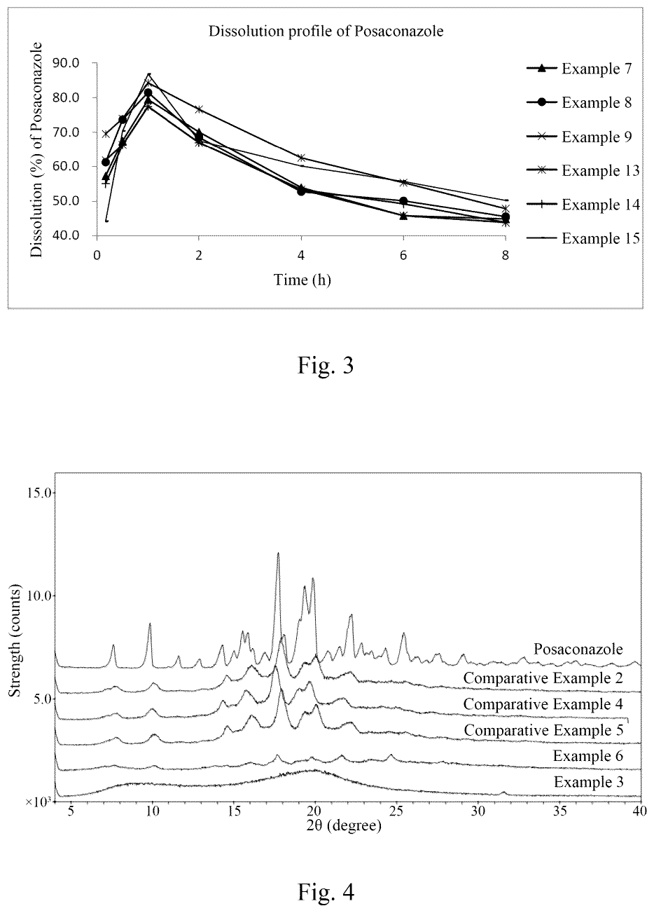

The dissolution profiles of posaconazole for the liquid compositions of Examples 3 to 6, and Comparative Examples 2, 4 and 5 were shown in FIG. 2. The dissolution profile of posaconazole for the liquid composition of Example 15 was shown in FIG. 3.

Conclusion: Combining FIGS. 2 and 3, it was evident that without the addition of the dilution stabilizer or other conventional crystallization inhibitors (e.g., povidone K30 or polyethylene glycol 4000), posaconazole in the liquid composition precipitated rapidly in media simulating intestinal pH, resulting in the significantly lower concentration of posaconazole dissolved in the solution.

Overall, where hydroxypropyl methylcellulose or hydroxypropyl cellulose was used as the dilution stabilizer, the liquid composition of posaconazole maintained a higher concentration of posaconazole dissolved in media simulating intestinal pH, and prolonged the duration of the dissolved state. During the initial release phase, a higher amount of hydroxypropyl methylcellulose as the dilution stabilizer resulted in a reduced release of posaconazole in the medium, likely due to the increased viscosity and slower dispersion of the liquid composition at the elevated concentration of hydroxypropyl methylcellulose. During the later release phase, hydroxypropyl methylcellulose as the dilution stabilizer inhibited posaconazole precipitation, thereby prolonging the duration of supersaturation of the solution.

Additionally, dissolution profiles of posaconazole for the liquid compositions of Examples 7 to 9, 13 and 14 were shown in FIG. 3.

Conclusion: Combining FIGS. 1 to 3, all of the liquid compositions in the above-mentioned Examples of the present disclosure maintained a high concentration of posaconazole dissolved in media simulating intestinal pH.

### 2. Stability Test

Refer to the “Content Determination Method” and “Related Substance Determination Method” in the aforementioned general determination methods.

The contents of posaconazole and related substances in each liquid composition and in the posaconazole active pharmaceutical ingredient were tested respectively. The contents of posaconazole and related substances in the liquid compositions of Examples 9 to 12 and Comparative Examples 1, 4 and 5 as well as in the posaconazole active pharmaceutical ingredient were shown in Table 3.

TABLE 3
Sample stability data

| | | | | Maximum | |
|---|---|---|---|---|---|
| | | | | Unknown | |
| | Placement | Placement | | Individual | Total |
| Sample Name | Condition | Time | | Impurity % | Impurities % |
| Posaconazole Active | — | | | 0.021 | 0.092 |
| Pharmaceutical | | | | | |
| Ingredient | | | | | |
| Comparative Example 1 | Day 0 | | | 0.025 | 0.043 |
| | Room temperature | 6 | months | 0.043 | 0.094 |
| | 40° C. | 1 | month | 0.048 | 0.229 |
| | | 3 | months | 0.105 | 0.441 |
| | 60° C. | 10 | days | 0.415 | 0.905 |
| | | 1 | month | 0.825 | 2.149 |
| Example 16 | Day 0 | | | 0.021 | 0.092 |
| | Room temperature | 6 | months | 0.043 | 0.126 |
| | 40° C. | 1 | month | 0.109 | 0.178 |
| | | 3 | months | 0.29 | 0.368 |
| | 60° C. | 10 | days | 0.472 | 0.623 |
| | | 1 | month | 1.037 | 1.193 |
| Example 17 | Day 0 | | | 0.037 | 0.096 |
| | Room temperature | 6 | months | 0.088 | 0.276 |
| | 40° C. | 1 | month | 0.082 | 0.374 |
| | | 3 | months | 0.149 | 1.036 |
| | 60° C. | 10 | days | 0.137 | 1.206 |
| | | 1 | month | 0.321 | 1.378 |
| Example 9 | Day 0 | | | 0.029 | 0.091 |
| | Room temperature | 6 | months | 0.065 | 0.254 |
| | 40° C. | 1 | month | 0.103 | 0.410 |
| | | 3 | months | 0.134 | 0.331 |
| | 60° C. | 10 | days | 0.166 | 0.443 |
| | | 1 | month | 0.266 | 0.637 |
| Example 10 | Day 0 | | | 0.018 | 0.099 |
| | Room temperature | 6 | months | 0.033 | 0.127 |
| | 40° C. | 1 | month | 0.057 | 0.225 |
| | | 3 | months | 0.088 | 0.376 |
| | 60° C. | 10 | days | 0.058 | 0.307 |
| | | 1 | month | 0.105 | 0.397 |
| Example 11 | Day 0 | | | 0.022 | 0.057 |
| | Room temperature | 6 | months | 0.02 | 0.052 |
| | 40° C. | 1 | month | 0.032 | 0.141 |
| | | 3 | months | 0.051 | 0.199 |
| | 60° C. | 10 | days | 0.026 | 0.188 |
| | | 1 | month | 0.079 | 0.203 |
| Example 12 | Day 0 | | | 0.017 | 0.078 |
| | Room temperature | 6 | months | 0.022 | 0.098 |
| | 40° C. | 1 | month | 0.04 | 0.108 |
| | | 3 | months | 0.045 | 0.204 |
| | 60° C. | 10 | days | 0.037 | 0.166 |
| | | 1 | month | 0.042 | 0.193 |

Conclusion: As shown in Table 3, the liquid compositions of Examples 9 to 12 of the present disclosure exhibited better stability during storage under both room temperature condition and accelerated condition, compared to those of Comparative Example 1 (without antioxidant) and Examples 16 and 17 (containing ascorbic acid or thioglycerol as the antioxidant). In particular, the liquid composition comprising methionine as the antioxidant exhibited superior stability during storage under both accelerated condition and room temperature condition, with a lower content of maximum individual impurity and/or total impurities.

### 3. Crystal Form Test

Following the “Crystal Form Determination Method” in the aforementioned general determination methods, the precipitates were collected upon completion of dissolution (i.e., after 480 minutes), combined and centrifugated at 12000 rpm for 5 minutes. The sediments were taken, air-dried and ground to fine powders, and the crystal form was determined using a DX-27mini X powder diffractometer.

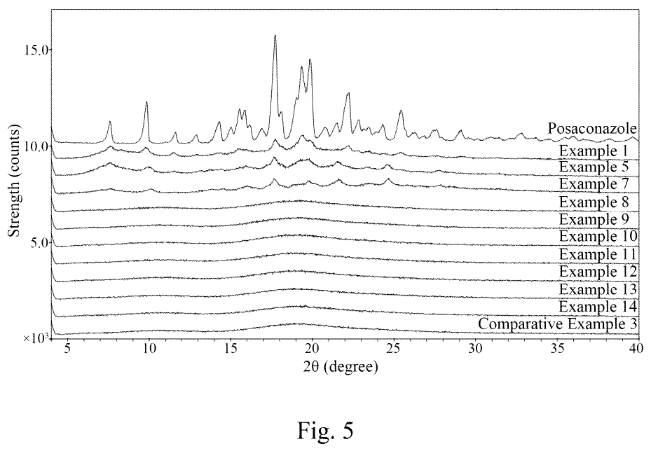

The test results were shown in FIGS. 4 and 5.

Conclusion: As shown in FIGS. 4 and 5, the precipitates following the dissolution test for the liquid compositions of Examples 1 to 2 and 5 to 14 of the present disclosure exhibited the following characteristics in the XRD patterns: the diffraction peak corresponding to posaconazole had a weaker intensity or was even absent, compared to Comparative Examples 2, 4 and 5, and posaconazole active pharmaceutical ingredient. This indicated that the amount of posaconazole present in crystalline form in the liquid compositions was low or even absent. In other words, posaconazole dissolved from the liquid composition of the present disclosure existed predominantly in a dissolved state or in amorphous form, with little or no crystalline precipitation occurring.

### 4. Pharmacokinetic Test

In the following pharmacokinetic studies, the doses administered to Beagle dogs were based on the amounts of posaconazole contained in the formulations. For example, intravenous infusion of 3 mg/kg of a posaconazole injection referred to intravenous infusion of the posaconazole injection at a dose of 3 mg posaconazole per kg of animal body weight. For another example, oral administration of 6 mg/kg of HYPS-04 referred to oral administration of HYPS-04 at a dose of 6 mg posaconazole per kg of animal body weight. For another example, oral administration of 10 mg/kg of a posaconazole enteric-coated tablet referred to oral administration of the posaconazole enteric-coated tablet at a dose of 10 mg posaconazole per kg of animal body weight.

### 4.1) Pharmacokinetic Study in Beagle Dogs Via Oral Administration of HYPS-04 and Intravenous Infusion of Posaconazole Injection

### Source of Samples and Controls:

| | |
|---|---|
| Name: Posaconazole Injection | Name: HYPS-04 |
| Batch No.: 231229 | Batch No.: PO-231228 (liquid |
| Specification: 300 mg | composition of Example 5) |
| Concentration: 300 mg/16.7 mL | Specification: 300 mg |
| Expiry date: Dec. 28, 2025 | Concentration: 20 mg/mL |
| Storage conditions: 2-8° C., | Expiry date: Dec. 27, 2025 |
| protected from light, tightly | Storage conditions: Room |
| closed | temperature, protected from |
| | light, tightly closed |

Objective: This study aimed to investigate pharmacokinetic parameters of HYPS-04 in Beagle dogs following oral administration and compared said pharmacokinetic parameters with those of the marketed posaconazole injection.

Method: 9 female Beagle dogs were divided into two groups. Group 1 (3 animals) received intravenous infusion of 3 mg/kg of the posaconazole injection, and blood samples were collected at 0 h prior to infusion, 1 min post infusion initiation and 3 min post infusion initiation, and 0 h post infusion completion, 0.083 h (about 5 min post-dosing), 0.25 h, 0.5 h, 1 h, 1.5 h, 2 h, 4 h, 6 h, 8 h, 12 h, 16 h, 24 h, 36 h, 48 h, 72 h, 96 h post infusion completion. Group 2 (6 animals) received oral administration of 6 mg/kg of HYPS-04 (i.e., the content of the active ingredient posaconazole in the orally administered HYPS-04 was twice that of the active ingredient posaconazole in the intravenously infused posaconazole injection), and blood samples were collected at 0.25 h, 0.5 h, 1 h, 2 h, 4 h, 6 h, 8 h, 10 h, 12 h, 14 h, 16 h, 24 h, 36 h, 48 h, 72 h, 96 h, 120 h, 144 h post-dosing.

Following centrifugation of whole blood to obtain plasma samples, the samples were analyzed to determine drug concentrations, and pharmacokinetic parameters were fitted using the DAS non-compartmental model.

### Results:

| Pharmacokinetic | | | |
|---|---|---|---|
| Parameters | Unit | Group 1 | Group 2 |
| AUC (0-t) | Ug/L*h | 30249.659 | 69280.060 |
| MRT (0-t) | H | 29.698 | 40.051 |
| T½z | H | 26.4 | 25.859 |
| Tₘₐₓ | H | 0.5 | 3.667 |
| Cₘₐₓ | Ug/L | 1257.001 | 1570.939 |

| | | |
|---|---|---|
| F | — | 114.51% |

Conclusion: The bioavailability of HYPS-04 following oral administration to Beagle dogs was 114.51%.

### 4.2) Pharmacokinetic Study in Beagle Dogs Via Oral Administration of HYPS-04 by Gavage and Oral Administration of Posaconazole Enteric-Coated Tablet

### Source of Samples and Controls:

| | | | |
|---|---|---|---|
| Name: Posaconazole | Name: HYPS-04 | Name: HYPS-04 | Name: HYPS-04 |
| enteric-coated tablet | Batch No.: PO- | Batch No.: PO- | Batch No.: PO- |
| Batch No.: A102025 | 240307 (liquid | 240322 (liquid | 240325 (liquid |
| Specification: 100 mg | composition of | composition of | composition of |
| Expiry date: Apr. 20, | Example 2) | Example 5) | Comparative Example 3) |
| 2025 | Specification: 300 mg | Specification: 300 mg | Specification: 300 mg |
| Storage conditions: | Concentration: | Concentration: | Concentration: |
| Store at 20-25° C., | 20 mg/mL | 20 mg/mL | 20 mg/mL |
| with an allowable | Expiry date: Mar. | Expiry date: Mar. | Expiry date: Mar. |
| deviation of 15-30° C. | 6, 2026 | 21, 2026 | 24, 2026 |
| Manufacturer: N.V. | Storage conditions: | Storage conditions: | Storage conditions: |
| Organon (The | Room temperature, | Room temperature, | Room temperature, |
| Netherlands) | protected from light, | protected from light, | protected from light, |
| | tightly closed | tightly closed | tightly closed |
| | Manufacturer: Hefei | Manufacturer: Hefei | Manufacturer: Hefei |
| | Cosoure | Cosoure | Cosoure |
| | Pharmaceuticals Inc. | Pharmaceuticals Inc. | Pharmaceuticals Inc. |

Objective: This study aimed to investigate the time course of plasma concentration of posaconazole in Beagle dogs following oral administration of the posaconazole enteric-coated tablets and oral administration of HYPS-04 by gavage, and to calculate the corresponding pharmacokinetic parameters.

Method: 24 Beagle dogs were randomly divided into 4 groups (6 dogs per group with equal numbers of males and females). Group 1 received oral administration of the posaconazole enteric-coated tablets; Group 2 received oral administration of HYPS-04 of Example 5; Group 3 received oral administration of HYPS-04 of Example 2; and Group 4 received oral administration of HYPS-04 of Comparative Example 3. Feeding protocol: feeding was delayed on the day of administration: one-third of the normal feeding amount was provided prior to dosing, dosing occurred within 20 minutes following completion of the feeding, and normal feeding resumed 4 hours post-dosing, and blood samples were collected at 0.25 h, 0.5 h, 1 h, 2 h, 4 h, 8 h, 12 h, 24 h, 48 h, 72 h, 96 h, 120 h, and 144 h post-dosing. Plasma samples were analyzed to determine the plasma concentration of posaconazole. The concentration of posaconazole in plasma samples was measured using the LC-MS/MS method.

Results: Following oral administration of 10 mg/kg of the posaconazole enteric-coated tablets to Beagle dogs, AUC₀₋₁₄₄ₕ of posaconazole was 108000 ng/mL*h, Cₘₐₓ was 2900 ng/mL, half-life was 23.30 h, and Tₘₐₓ was 4.00 h in plasma of male dogs, and AUC₀₋₁₄₄ₕ of posaconazole was 109000 ng/mL*h, Cₘₐₓ was 2170 ng/mL, half-life was 29.70 h, and Tₘₐₓ was 6.67 h in plasma of female dogs.

Following oral administration of 10 mg/kg of the HYPS-04 of Example 5 to Beagle dogs, AUC₀₋₁₄₄ₕ of posaconazole was 118600 ng/mL*h, Cₘₐₓ was 2570 ng/mL, half-life was 25.30 h, and Tₘₐₓ was 4.00 h in plasma of male dogs, and AUC₀₋₁₄₄ₕ of posaconazole was 125000 ng/mL*h, Cₘₐₓ was 3050 ng/mL, half-life was 29.10 h, and Tₘₐₓ was 1.50 h in plasma of female dogs.

Following oral administration of 10 mg/kg of the HYPS-04 of Example 2 to Beagle dogs, AUC₀₋₁₄₄ₕ of posaconazole was 97650 ng/mL*h, Cₘₐₓ was 2260 ng/mL, half-life was 20.50 h, and Tₘₐₓ was 3.50 h in plasma of male dogs, and AUC₀₋₁₄₄ₕ of posaconazole was 97600 ng/mL*h, Cₘₐₓ was 2117 ng/mL, half-life was 22.66 h, and Tₘₐₓ was 2.50 h in plasma of female dogs.

Following oral administration of 10 mg/kg of the HYPS-04 of Comparative Example 3 to Beagle dogs, AUC₀₋₁₄₄ₕ of posaconazole was 64363 ng/mL*h, Cₘₐₓ was 1321 ng/mL, half-life was 19.87 h, and Tₘₐₓ was 2.00 h in plasma of male dogs, and AUC₀₋₁₄₄ₕ of posaconazole was 70123 ng/mL*h, Cₘₐₓ was 1223 ng/mL, and half-life was 22.12 h, Tₘₐₓ was 2.50 h in plasma of female dogs.

Conclusion: The bioavailability calculated on the basis of the intravenous administration results of 2024-PK-002 was as follows: the bioavailability of orally administered enteric-coated tablets in animals of Group 1 was 59.4% (male) and 60.0% (female), respectively; the bioavailability of orally administered HYPS-04 of Example 5 in animals of Group 2 was 65.3% (male) and 68.8% (female), respectively; the bioavailability of orally administered HYPS-04 of Example 2 in animals of Group 3 was 53.7% (male), 53.7% (female), respectively; and the bioavailability of orally administered HYPS-04 of Comparative Example 3 in animals of Group 4 was 35.4% (male), 38.6% (female), respectively.

Compared to the enteric-coated tablets in Group 1, the relative bioavailability in animals of Group 2 was 103.37% (male) and 114.7% (female); the relative bioavailability in animals of Group 3 was 90.42% (male) and 89.50% (female); the relative bioavailability in animals of Group 4 was 59.6% (male) and 64.33% (female).

### 4.3) Pharmacokinetic Study in Beagle Dogs in Fed and Fasted States Via Oral Administration of HYPS-04 by Gavage and Oral Administration of Posaconazole Enteric-Coated Tablets

### Source of Samples and Controls:

| | | |
|---|---|---|
| Name: Posaconazole enteric- | Name: HYPS-04 | Name: HYPS-04 |
| coated tablets | Batch No.: PO-240307 | Batch No.: PO-240429 |
| Batch No.: A102025 | (liquid composition of | (liquid composition of |
| Specification: 100 mg | Example 2) | Example 5) |
| Expiry date: Apr. 20, 2025 | Specification: 300 mg | Specification: 300 mg |
| Storage conditions: Store at | Concentration: 20 mg/mL | Concentration: 20 mg/mL |
| 20-25° C., with an allowable | Expiry date: Mar. 6, 2026 | Expiry date: Apr. 20, 2026 |
| deviation of 15-30° C. | Storage conditions: Room | Storage conditions: Room |
| Manufacturer: N.V. Organon | temperature, protected from | temperature, protected from |
| (The Netherlands) | light, tightly closed | light, tightly closed |
| | Manufacturer: Hefei Cosoure | Manufacturer: Hefei Cosoure |
| | Pharmaceuticals Inc. | Pharmaceuticals Inc. |

Objective: This study aimed to investigate the time course of plasma concentration of posaconazole in Beagle dogs in a fed state and in a fasted state following oral administration of posaconazole enteric-coated tablets and oral administration of HYPS-04 by gavage, to calculate the corresponding pharmacokinetic parameters, and to compare exposure differences.

Method: Nine healthy and suitable male dogs were divided into three groups with three dogs per group, and were administered with the posaconazole enteric tablets, HYPS-04 of Example 5 and HYPS-04 of Example 2, respectively. Cycle I: the dogs were fasted for 12 hours at each pre-dosing, Group 1 received the posaconazole enteric-coated tablets, Group 2 received HYPS-04 of Example 5, and Group 3 received HYPS-04 of Example 2. Cycle II: the dogs were fed before dosing, then dosed within 20 minutes after feeding completed, Group 1 received the posaconazole enteric-coated tablets, Group 2 received HYPS-04 of Example 5 and Group 3 received HYPS-04 of Example 2. Cycle III: the dogs were fasted for 12 hours at each pre-dosing, Group 1 received HYPS-04 of Example 5, and Group 2 received the posaconazole enteric-coated tablets. Cycle IV: the dogs were fed before dosing, then dosed within 20 minutes after feeding completed, Group 1 received HYPS-04 of Example 5, and Group 2 received the posaconazole enteric-coated tablets. Each cycle was separated by a one-week washout period. Blood samples were collected at 0.25 h, 0.5 h, 1 h, 2 h, 4 h, 8 h, 12 h, 24 h, 48 h, 72 h, 96 h, 120 h, and 144 h post-dosing. Plasma samples were analyzed to determine the plasma concentration of posaconazole. The concentration of posaconazole in plasma samples was measured using the LC-MS/MS method.

Results: Following oral administration of 10 mg/kg of the posaconazole enteric-coated tablets to Beagle dogs, AUC₀₋₁₄₄ₕ of posaconazole was 70200 ng/mL*h, Cₘₐₓ was 1290 ng/mL, half-life was 31.60 h, and Tₘₐₓ was 6.00 h in plasma of dogs in the fasted state, while AUC₀₋₁₄₄ₕ of posaconazole was 141000 ng/mL*h, Cₘₐₓ was 2270 ng/mL, and half-life was 33.90 h, and Tₘₐₓ was 8.00 h in plasma of dogs in the fed state.

Following oral administration of 10 mg/kg of the HYPS-04 of Example 5 to Beagle dogs by gavage, AUC₀₋₁₄₄ₕ of posaconazole was 128400 ng/mL*h, Cₘₐₓ was 2130 ng/mL, half-life was 31.80 h, and Tₘₐₓ was 8.00 h in plasma of dogs in the fasted state, while AUC₀₋₁₄4 h of posaconazole was 127000 ng/mL*h, Cₘₐₓ was 2260 ng/mL, and half-life was 31.30 h, and Tₘₐₓ was 8.67 h in plasma of dogs in the fed state.

Following oral administration of 10 mg/kg of the HYPS-04 of Example 2 to Beagle dogs by gavage, AUC₀₋₁₄₄ₕ of posaconazole was 95620 ng/mL*h, Cₘₐₓ was 2010 ng/mL, half-life was 25.90 h, and Tₘₐₓ was 6.00 h in plasma of dogs in the fasted state, while AUC₀₋₁₄₄ₕ of posaconazole was 97000 ng/mL*h, Cₘₐₓ was 2060 ng/mL, and half-life was 30.60 h, and Tₘₐₓ was 8.00 h in plasma of dogs in the fed state.

Conclusion: The bioavailability calculated on the basis of the intravenous administration results of 2024-PK-002 was as follows: the bioavailability of orally administered enteric-coated tablets in dogs in the fasted state was 34.6%; the bioavailability of orally administered HYPS-04 of Example 5 by gavage in dogs in the fasted state was 70.6%; the bioavailability of orally administered HYPS-04 of Example 2 by gavage in dogs in the fasted state was 52.6%; the bioavailability of orally administered enteric-coated tablets in dogs in the fed state was 77.6%; the bioavailability of orally administered HYPS-04 of Example 5 by gavage in dogs in the fed state was 69.9%; and the bioavailability of orally administered HYPS-04 of Example 2 by gavage in dogs in the fed state was 53.4%. This indicated that the bioavailability of the liquid compositions of posaconazole in Example 2 and Example 5 was not affected by food, whereas the bioavailability of the oral enteric-coated tablets was markedly affected by food.

What is claimed is:

1. A liquid composition comprising posaconazole, a cyclodextrin, a dilution stabilizer, an antioxidant, an acidic pH regulator and water, wherein the dilution stabilizer comprises hydroxypropyl cellulose and/or hydroxypropyl methylcellulose, and the cyclodextrin is present in an amount of 25% to 45% (w/v), based on the total volume of the liquid composition.

2. The liquid composition of claim 1, wherein a mass ratio of posaconazole to the dilution stabilizer is 1:(1 to 7.5), or 1:(1.5 to 4), or 1:(2 to 3); and/or
a mass ratio of posaconazole to the cyclodextrin is 1:(10 to 22.5), or 1:(11 to 20), or 1:(12.5 to 17.5).

3. The liquid composition of claim 1, wherein the antioxidant comprises one or more of methionine, sodium metabisulfite, sodium bisulfite, proline, and glycine; or
the antioxidant comprises methionine; or
the antioxidant comprises methionine and one or more of sodium metabisulfite, sodium bisulfite, proline, and glycine.

4. The liquid composition of claim 2, wherein the antioxidant comprises one or more of methionine, sodium metabisulfite, sodium bisulfite, proline, and glycine; or
the antioxidant comprises methionine; or
the antioxidant comprises methionine and one or more of sodium metabisulfite, sodium bisulfite, proline, and glycine.

5. The liquid composition of claim 1, wherein the cyclodextrin comprises β-cyclodextrin and/or a derivative of β-cyclodextrin; or
the cyclodextrin comprises one or more of hydroxypropyl-β-cyclodextrin, sulfobutyl ether-β-cyclodextrin, and methyl-β-cyclodextrin.

6. The liquid composition of claim 2, wherein the cyclodextrin comprises β-cyclodextrin and/or a derivative of β-cyclodextrin; or
the cyclodextrin comprises one or more of hydroxypropyl-β-cyclodextrin, sulfobutyl ether-β-cyclodextrin, and methyl-β-cyclodextrin.

7. The liquid composition of claim 4, wherein the cyclodextrin comprises β-cyclodextrin and/or a derivative of β-cyclodextrin; or
the cyclodextrin comprises one or more of hydroxypropyl-β-cyclodextrin, sulfobutyl ether-β-cyclodextrin, and methyl-β-cyclodextrin.

8. The liquid composition of claim 1, wherein the acidic pH regulator comprises one or more of hydrochloric acid, sulfuric acid, phosphoric acid, and maleic acid; or
the acidic pH regulator comprises hydrochloric acid and/or sulfuric acid; and/or
wherein a pH value of the liquid composition is 1.5 to 4 or 2 to 3.

9. The liquid composition of claim 2, wherein the acidic pH regulator comprises one or more of hydrochloric acid, sulfuric acid, phosphoric acid, and maleic acid; or
the acidic pH regulator comprises hydrochloric acid and/or sulfuric acid; and/or
wherein a pH value of the liquid composition is 1.5 to 4 or 2 to 3.

10. The liquid composition of claim 4, wherein the acidic pH regulator comprises one or more of hydrochloric acid, sulfuric acid, phosphoric acid, and maleic acid; or
the acidic pH regulator comprises hydrochloric acid and/or sulfuric acid; and/or
wherein a pH value of the liquid composition is 1.5 to 4 or 2 to 3.

11. The liquid composition of claim 1, wherein posaconazole is present in an amount of 1% to 2.7% (w/v), 1.5% to 2.5% (w/v), or 1.5% to 2.0% (w/v); the cyclodextrin is present in an amount of 25% to 45% (w/v), 25% to 40% (w/v), or 25% to 35% (w/v); the dilution stabilizer is present in an amount of 2% to 15% (w/v), 3% to 8% (w/v), or 4% to 6% (w/v); the antioxidant is present in an amount of 0.05% to 2% (w/v); and the acidic pH regulator is present in an amount of 1.5% to 4% (w/v), based on the total volume of the liquid composition.

12. The liquid composition of claim 2, wherein posaconazole is present in an amount of 1% to 2.7% (w/v), 1.5% to 2.5% (w/v), or 1.5% to 2.0% (w/v); the cyclodextrin is present in an amount of 25% to 45% (w/v), 25% to 40% (w/v), or 25% to 35% (w/v); the dilution stabilizer is present in an amount of 2% to 15% (w/v), 3% to 8% (w/v), or 4% to 6% (w/v); the antioxidant is present in an amount of 0.05% to 2% (w/v); and the acidic pH regulator is present in an amount of 1.5% to 4% (w/v), based on the total volume of the liquid composition.

13. The liquid composition of claim 4, wherein posaconazole is present in an amount of 1% to 2.7% (w/v), 1.5% to 2.5% (w/v), or 1.5% to 2.0% (w/v); the cyclodextrin is present in an amount of 25% to 45% (w/v), 25% to 40% (w/v), or 25% to 35% (w/v); the dilution stabilizer is present in an amount of 2% to 15% (w/v), 3% to 8% (w/v), or 4% to 6% (w/v); the antioxidant is present in an amount of 0.05% to 2% (w/v); and the acidic pH regulator is present in an amount of 1.5% to 4% (w/v), based on the total volume of the liquid composition.

14. The liquid composition of claim 1, further comprising one or more of a flavoring agent, a preservative, and a chelating agent.

15. The liquid composition of claim 2, further comprising one or more of a flavoring agent, a preservative, and a chelating agent.

16. The liquid composition of claim 14, wherein,
the flavoring agent comprises a sweetener and/or a flavor; and/or
the preservative comprises one or more of methylparaben, ethylparaben, and propylparaben; and/or
the chelating agent comprises one or more of citric acid, malic acid, benzoic acid, edetic acid, sodium edetate; and/or
the flavoring agent is present in an amount of 0.1% to 10% (w/v), and/or the preservative is present in an amount of 0.01% to 1% (w/v), and/or the chelating agent is present in an amount of 0.01% to 0.1% (w/v), based on the total volume of the liquid composition.

17. The liquid composition of claim 15, wherein,
the flavoring agent comprises a sweetener and/or a flavor; and/or
the preservative comprises one or more of methylparaben, ethylparaben, and propylparaben; and/or
the chelating agent comprises one or more of citric acid, malic acid, benzoic acid, edetic acid, sodium edetate; and/or
the flavoring agent is present in an amount of 0.1% to 10% (w/v), and/or the preservative is present in an amount of 0.01% to 1% (w/v), and/or the chelating agent is present in an amount of 0.01% to 0.1% (w/v), based on the total volume of the liquid composition.

18. The liquid composition of claim 12, further comprising one or more of a flavoring agent, a preservative, and a chelating agent, wherein,
the flavoring agent comprises a sweetener and/or a flavor; and/or
the preservative comprises one or more of methylparaben, ethylparaben, and propylparaben; and/or
the chelating agent comprises one or more of citric acid, malic acid, benzoic acid, edetic acid, sodium edetate; and/or
the flavoring agent is present in an amount of 0.1% to 10% (w/v), and/or the preservative is present in an amount of 0.01% to 1% (w/v), and/or the chelating agent is present in an amount of 0.01% to 0.1% (w/v), based on the total volume of the liquid composition.

19. A method for preparing the liquid composition of claim 1, comprising:
mixing the cyclodextrin with an aqueous solvent to obtain a first mixture;
mixing posaconazole, the acidic pH regulator and the antioxidant with the first mixture to obtain a second mixture;
mixing the dilution stabilizer with the second mixture to obtain a third mixture;
and adding the aqueous solvent to the third mixture to make up the volume.

20. A method for treating or ameliorating a fungal infection in a subject in need thereof, comprising administering the liquid composition of claim 1 to the subject.
